(19) United States
(12) Patent Application Publication
(10) Pub. No.: US 2015/0011009 A1
(43) Pub. Date: Jan. 8, 2015

(54) Method and Apparatus For Determining A Calorific Value Parameter, As Well As A Gas-Powered System Comprising Such An Apparatus

(71) Applicant: Karl Dungs GmbH & Co. KG, Urbach (DE)

(72) Inventors: Oliver Schmidt, Neuenstadt (DE); Harald Petermann, Lichtenwald (DE); Siegfried Berger, Schlierbach (DE); Rudolf Haug, Apen (DE)

(73) Assignee: Karl Dungs GmbH & Co. KG, Urbach (DE)

(21) Appl. No.: 14/315,832

(22) Filed: Jun. 26, 2014

(30) Foreign Application Priority Data
Jul. 3, 2013 (DE) 102013106987.8

(51) Int. Cl. G01N 33/22

(52) U.S. Cl. CPC G01N 33/225
USPC 436/160; 422/51

(57) ABSTRACT

A method and an apparatus for determining the calorific value parameter describing the calorific value of a gaseous fuel. The apparatus comprises a test burner with a test combustion chamber. An air ratio sensor is arranged in an exhaust gas duct of the test burner and measures an air ratio signal that corresponds to the air ratio of the exhaust gas. As a function of the received air ratio signal, at least one setting signal is generated for a test supply unit via a test control unit. The setting signal controls the amount and/or the proportion of a gaseous fuel or an oxygen-containing gas that is supplied to the test combustion chamber. A calorific value sensor arrangement is provided in the combustion chamber and has an ionization sensor and, a temperature sensor. The sensor signal of the calorific value sensor is transmitted to a determination unit.

### CROSS REFERENCE TO RELATED APPLICATIONS

The present application is based upon and claims the benefit of German application 10 2013 106 987.8, filed 3 Jul. 2013.

### BACKGROUND OF THE INVENTION

The invention relates to a method and an apparatus for determining a calorific value parameter for the calorific value of a gaseous fuel. Furthermore, the invention relates to a gas-powered system comprising an apparatus for determining the calorific value parameter.

As a rule, burners of gas-powered systems are adjusted in such a manner that a complete combustion of a supplied gaseous fuel does take place. To accomplish this, the amount of supplied fuel, as well as the amount of supplied oxygen-containing gas—in particular air, is adjusted accordingly. As a rule, a sensor for determining the air ratio is provided in the exhaust air of such a burner, so that a stoichiometric or super-stoichiometric combustion takes place.

A gaseous fuel, for example biofuel, natural gas, hydrogen or a gas mixture of several such gases, exhibits different calorific values. Frequently, gas-powered systems must be operated with different gases or gas mixtures that may also vary during operation. Therefore, as a rule, the proportion of methane in natural gas fluctuates between 75% and approximately 95% to 98%. In addition, natural gas may contain varying proportions of ethane, propane, butane, nitrogen or carbon dioxide. As has already been mentioned, it is also possible to use gas mixtures as a gaseous fuel, so that it is possible that biogas or proportions of biogas, as well as hydrogen or proportions of hydrogen, may be present. In doing so, the calorific value varies accordingly.

In order to operate a gas-powered system it may thus be advantageous to perform any adjustment as a function of the gas or gas mixture that is being used.

To this end, various apparatus and systems have been suggested. Document EP 199 55 76 A1 suggests a system comprising a Fabry-Perot interferometer for the detection of substances or substance concentrations in the gaseous fuel. The system as in DE 103 02 487 A1 uses an infrared absorption gas metering system comprising at least two measuring channels for carbon dioxide and alkalis in order to determine the fuel gas composition.

Document U.S. Pat. No. 4,118,172 A suggests the use of a test burner comprising an air ratio sensor for adjusting a stoichiometric combustion and to then transfer this adjustment to a main burner. In this case, there is no determination of a calorific value parameter.

Document DE 699 24 828 T2 describes the determination of the calorific value as a function of the measurement of the speed of sound at various temperatures.

In accordance with document DE 101 298 08 a calorific value is determined in that a fuel/air mixture is combusted and subsequently cooled. In doing so, the temperature is measured at various points and a heat flow is calculated, whereby said heat flow can be used for the determination of the calorific value.

The method disclosed in document DE 100 10 291 A1 pursues a similar approach. In this case, a heat transfer element is installed in the combustion chamber and sufficient heat is withdrawn from the heat transfer element to achieve a constant temperature of the heat transfer element. Based on this, it is then concluded that the heat output generated by the burner corresponds to the heat output that was withdrawn from the heat transfer element. This heat output is then used to determine the calorific value.

Document EP 1 770 390 A1 describes another method and a device for determining the Wobbe index and the calorific value, respectively. In this case, two heating panels are arranged in a chamber. In doing so, the second heating panel acts as the catalytic heating panel. A control device is used to keep the temperature of at least one of the two heating panels constant. When the gas/air mixture flows past the first heating panel, the output necessary to maintain this heating panel at a constant temperature is determined. The gas/air mixture can be combusted in the region of the other heating panel. At the same time, a determination is made as to what output is necessary in order to also keep the second heating panel at a constant temperature. These two output values can be used to determine a calorific value for temperature regulation.

Considering this, it may be viewed as the object of the present invention that an apparatus and a method be created, wherein the apparatus requires only simple means and permits a fast and safe determination of the calorific value.

In accordance with the invention a test burner comprising a test combustion chamber is used therefor. An exhaust gas duct leads out of the test combustion chamber, wherein an air ratio sensor is arranged in said gas duct. The air ratio sensor produces an air ratio signal that indicates the air ratio in the exhaust gas of the test combustion chamber. The air ratio signal is transmitted to a test control unit. The test control unit generates a setting signal as a function of the air ratio signal. A test supply unit is disposed to supply the gaseous fuel together with an oxygen-containing gas, for example air, to the test combustion chamber. The test supply unit can be controlled and can thus adjust the proportion and/or the amounts of the gaseous fuel supplied to the test combustion chamber and/or the oxygen-containing gas introduced into the test combustion chamber. In doing so, the volume flow of the mass flow of the oxygen-containing gas and/or the gaseous fuel through the test supply unit is adjusted as a function of the at least one setting signal. As a result of this, a combustion or an oxidation of the mixture of the fuel and the oxygen-containing gas can be adjusted in such a manner that the air ratio corresponds to a prespecified air ratio setpoint value.

A calorific value sensor arrangement is provided in the region of the flame or in the region of the oxidation of the fuel-containing gas in the test combustion chamber. The calorific value sensor arrangement comprises at least one ionization sensor that generates an ionization signal and, in particular, an ionization current. The calorific value sensor arrangement may comprise additional sensors, for example, at least one temperature sensor and/or at least one optical sensor.

In addition, the apparatus comprises a determination unit. This determination unit is supplied with the at least one sensor signal of the calorific value arrangement and thus with the ionization signal. Depending on the ionization signal, a calorific value parameter is determined, said value parameter indicating the calorific value of the gaseous fuel. To do so, the ionization signal or the ionization current is determined at least one, or at several, prespecified air ratio values. The ionization in the test combustion chamber at a prespecified air ratio is characteristic of the heating value. This can be used to derive the calorific value parameter in the determination unit.

In order to be able to improve the accuracy of determination of the calorific value parameter, additional parameters or signals may be taken into consideration during the determination of the calorific value parameter, i.e., in particular one or more of the following parameters:
at least one parameter characterizing the burner line, for example, the at least one setting signal for adjusting the test supply arrangement;
a temperature value inside the test combustion chamber; to do so, the calorific value sensor arrangement may comprise a temperature sensor;
potentially a signal of an optical sensor of the calorific value sensor arrangement, by means of which a spectrum or a part of a spectrum of the light emitted by the flame of the test burner is determined.

Preferably, the calorific value parameter is determined in the determination unit based on the temperature signal of a temperature sensor, on the ionization signal and on the at least one setting signal of the test control unit. This triple value can be used for a sufficiently exact determination of the calorific value parameter, wherein the apparatus can function with very simple means. Additional sensor types for the spectral analysis, for the optical analysis or for measuring the speed of sound are not necessary. Inasmuch as the performance of the test burner influences the ionization, it is possible—in particular—to use the already provided at least one setting signal in a very simple manner for evaluating the burner performance. Indeed, it is the latter that is used to determine the amount of fuel and/or air that is introduced into the test combustion chamber. Alternatively, it is also possible to determine other parameters that characterize the burner performance.

Considering one embodiment, the determination unit may comprise a memory in which a determination specification is stored for determining the calorific value parameter. For example, the determination specification may be a table, a function, a characteristic map or the like. In order to minimize the required computation effort, preferably a characteristic map is stored in the memory, in which case, depending on the considered input signals or parameters, the calorific value parameter can be determined simply and rapidly during the operation of the test burner.

A test flame can be generated in the test combustion chamber via an ignition device, e.g., an ignition electrode. Alternatively, it is also possible to generate an oxidation without flame, for example with the aid of a catalyst. Preferably, the calorific value sensor arrangement is arranged in the region of the test flame.

Considering one exemplary embodiment, the calorific value sensor device may also comprise several ionization sensors that are located at various locations inside the test combustion chamber. The ionization, and thus the ionization signal, are a function of the position of the ionization sensor in the region of the test flame. By arranging several ionization sensors at different locations, it is possible to achieve an additional increase of accuracy in the determination of the calorific value parameter.

The apparatus for determining the calorific value parameter may be a component of a gas-powered system. A part of the gaseous fuel used for the gas-powered system is supplied to the test burner and its calorific value is determined as explained hereinabove.

The gas-powered system may comprise one main combustion chamber, one main supply arrangement and one main control unit. The main supply arrangement is disposed to adjust the amount of the gaseous fuel and/or the amount of the oxygen-containing gas that is introduced into the main combustion chamber. At least one main setting signal is generated via the main control unit for the actuation of the main supply arrangement. This main setting signal is generated in the main control unit as a function of the calorific value parameter determined in the apparatus for the determination of the calorific value parameters. Consequently, the main burner of the gas-powered system can be controlled and adjusted as a function of the calorific value parameter.

In particular, the operation of the main burner is to be started or made possible only when—via the device—a calorific value parameter for the currently used gaseous fuel was previously determined. It is only after the main control unit receives an actual value for the calorific value parameter that said control unit will actuate the main supply arrangement as a function of the latter and will start up the operation of the main burner. As a result of this, unfavorable operating conditions when the main burner is started can be prevented. Depending on the constituents of the gaseous fuel, it is also possible for explosive ignitions and dangerous operating conditions to occur when the mixtures are too rich when the burner is being started. In accordance with the invention such conditions are prevented in that, with the test burner, a calorific value parameter for the supplied gaseous fuel is available via the apparatus.

Preferably, the main supply unit is additionally adjusted as a function of a control signal. The control signal can be manually prespecified by the operator or be automatically prespecified by a governor or another arrangement. The performance of the main burner can be adjusted via the control signal.

Furthermore, it is advantageous if the currently determined value of the fuel parameter is saved in memory, for example in a memory of the determination device. After shutting down, this value is available for the renewed start of the test burner and/or the main burner. Consequently, this value can act as the starting parameter as long as the test burner still has not determined a more current value for the calorific value parameter.

Advantageous embodiments of the invention become obvious from the dependent patent claims as well as from the description. The description is restricted to the essential features of the invention. The drawings are to be used for supplementary information. Hereinafter, the exemplary embodiments of the invention will be explained in detail with reference to the attached drawings. They show in

### SUMMARY OF THE INVENTION

Considering this, it may be viewed as the object of the present invention that an apparatus and a method be created, wherein the apparatus requires only simple means and permits a fast and safe determination of the calorific value.

In accordance with the invention a test burner comprising a test combustion chamber is used therefor. An exhaust gas duct leads out of the test combustion chamber, wherein an air ratio sensor is arranged in said gas duct. The air ratio sensor produces an air ratio signal that indicates the air ratio in the exhaust gas of the test combustion chamber. The air ratio signal is transmitted to a test control unit. The test control unit generates a setting signal as a function of the air ratio signal. A test supply unit is disposed to supply the gaseous fuel together with an oxygen-containing gas, for example air, to the test combustion chamber. The test supply unit can be controlled and can thus adjust the proportion and/or the amounts of the gaseous fuel supplied to the test combustion chamber and/or the oxygen-containing gas introduced into the test combustion chamber. In doing so, the volume flow of the mass flow of the oxygen-containing gas and/or the gaseous fuel through the test supply unit is adjusted as a function of the at least one setting signal. As a result of this, a combustion or an oxidation of the mixture of the fuel and the oxygen-containing gas can be adjusted in such a manner that the air ratio corresponds to a prespecified air ratio setpoint value.

A calorific value sensor arrangement is provided in the region of the flame or in the region of the oxidation of the fuel-containing gas in the test combustion chamber. The calorific value sensor arrangement comprises at least one ionization sensor that generates an ionization signal and, in particular, an ionization current. The calorific value sensor arrangement may comprise additional sensors, for example, at least one temperature sensor and/or at least one optical sensor.

In addition, the apparatus comprises a determination unit. This determination unit is supplied with the at least one sensor signal of the calorific value arrangement and thus with the ionization signal. Depending on the ionization signal, a calorific value parameter is determined, said value parameter indicating the calorific value of the gaseous fuel. To do so, the ionization signal or the ionization current is determined at least one, or at several, prespecified air ratio values. The ionization in the test combustion chamber at a prespecified air ratio is characteristic of the heating value. This can be used to derive the calorific value parameter in the determination unit.

In order to be able to improve the accuracy of determination of the calorific value parameter, additional parameters or signals may be taken into consideration during the determination of the calorific value parameter, i.e., in particular one or more of the following parameters:
at least one parameter characterizing the burner line, for example, the at least one setting signal for adjusting the test supply arrangement;
a temperature value inside the test combustion chamber; to do so, the calorific value sensor arrangement may comprise a temperature sensor;
potentially a signal of an optical sensor of the calorific value sensor arrangement, by means of which a spectrum or a part of a spectrum of the light emitted by the flame of the test burner is determined.

Preferably, the calorific value parameter is determined in the determination unit based on the temperature signal of a temperature sensor, on the ionization signal and on the at least one setting signal of the test control unit. This triple value can be used for a sufficiently exact determination of the calorific value parameter, wherein the apparatus can function with very simple means. Additional sensor types for the spectral analysis, for the optical analysis or for measuring the speed of sound are not necessary. Inasmuch as the performance of the test burner influences the ionization, it is possible—in particular—to use the already provided at least one setting signal in a very simple manner for evaluating the burner performance. Indeed, it is the latter that is used to determine the amount of fuel and/or air that is introduced into the test combustion chamber. Alternatively, it is also possible to determine other parameters that characterize the burner performance.

Considering one embodiment, the determination unit may comprise a memory in which a determination specification is stored for determining the calorific value parameter. For example, the determination specification may be a table, a function, a characteristic map or the like. In order to minimize the required computation effort, preferably a characteristic map is stored in the memory, in which case, depending on the considered input signals or parameters, the calorific value parameter can be determined simply and rapidly during the operation of the test burner.

A test flame can be generated in the test combustion chamber via an ignition device, e.g., an ignition electrode. Alternatively, it is also possible to generate an oxidation without flame, for example with the aid of a catalyst. Preferably, the calorific value sensor arrangement is arranged in the region of the test flame.

Considering one exemplary embodiment, the calorific value sensor device may also comprise several ionization sensors that are located at various locations inside the test combustion chamber. The ionization, and thus the ionization signal, are a function of the position of the ionization sensor in the region of the test flame. By arranging several ionization sensors at different locations, it is possible to achieve an additional increase of accuracy in the determination of the calorific value parameter.

The apparatus for determining the calorific value parameter may be a component of a gas-powered system. A part of the gaseous fuel used for the gas-powered system is supplied to the test burner and its calorific value is determined as explained hereinabove.

The gas-powered system may comprise one main combustion chamber, one main supply arrangement and one main control unit. The main supply arrangement is disposed to adjust the amount of the gaseous fuel and/or the amount of the oxygen-containing gas that is introduced into the main combustion chamber. At least one main setting signal is generated via the main control unit for the actuation of the main supply arrangement. This main setting signal is generated in the main control unit as a function of the calorific value parameter determined in the apparatus for the determination of the calorific value parameters. Consequently, the main burner of the gas-powered system can be controlled and adjusted as a function of the calorific value parameter.

In particular, the operation of the main burner is to be started or made possible only when—via the device—a calorific value parameter for the currently used gaseous fuel was previously determined. It is only after the main control unit receives an actual value for the calorific value parameter that said control unit will actuate the main supply arrangement as a function of the latter and will start up the operation of the main burner. As a result of this, unfavorable operating conditions when the main burner is started can be prevented. Depending on the constituents of the gaseous fuel, it is also possible for explosive ignitions and dangerous operating conditions to occur when the mixtures are too rich when the burner is being started. In accordance with the invention such conditions are prevented in that, with the test burner, a calorific value parameter for the supplied gaseous fuel is available via the apparatus.

Preferably, the main supply unit is additionally adjusted as a function of a control signal. The control signal can be manually prespecified by the operator or be automatically prespecified by a governor or another arrangement. The performance of the main burner can be adjusted via the control signal.

Furthermore, it is advantageous if the currently determined value of the fuel parameter is saved in memory, for example in a memory of the determination device. After shutting down, this value is available for the renewed start of the test burner and/or the main burner. Consequently, this value can act as the starting parameter as long as the test burner still has not determined a more current value for the calorific value parameter.

Advantageous embodiments of the invention become obvious from the dependent patent claims as well as from the description. The description is restricted to the essential features of the invention. The drawings are to be used for supplementary information. Hereinafter, the exemplary embodiments of the invention will be explained in detail with reference to the attached drawings. They show in

### IN THE DRAWINGS

FIG. 1 a block circuit diagram of an exemplary embodiment of an apparatus for the determination of a calorific value parameter with the use of a test burner;

FIG. 2 a block circuit diagram of an exemplary embodiment of a gas-powered apparatus comprising a main burner;

FIG. 3 a schematic representation of the temperature T as a function of the distance z from an inlet into the test combustion chamber of the apparatus as in FIG. 1;

FIG. 4 a schematic representation of the dependence of the ionization signal I on the burner performance P of the test burner for various air ratios λ, and

FIG. 5 a schematic representation of the dependence of the ionization signal I on the burner performance P for various calorific values or compositions of the gaseous fuel.

### DETAILED DESCRIPTION OF THE INVENTION

FIG. 1 shows an exemplary embodiment of an apparatus 10 for determining a calorific value parameter H in the block circuit diagram. The calorific value parameter H indicates the calorific value of a gaseous fuel G. The gaseous fuel G may be, for example, a biogas, natural gas, hydrogen or a mixture of several of the mentioned gas constituents. Frequently, the exact composition of the gaseous fuel G and hence its calorific value are unknown. The apparatus 10 allows the determination of a calorific value parameter H, said parameter defining the calorific value.

To accomplish this, the apparatus 10 comprises a test burner 11 with a test combustion chamber 12. An inlet 14 terminates in the test combustion chamber 12 on a first side 13. Through the first inlet 14, gaseous fuel G is introduced via a test supply arrangement 15 into the combustion chamber 12.

In addition, a second inlet 16 terminates in the test combustion chamber 12 on the first side 13, by way of which inlet an oxygen-containing gas, for example air L, is introduced into the test combustion chamber 12 via the test supply arrangement 15. In the exemplary embodiment, the two inlets 14, 16 are arranged coaxially relative to each other. In doing so, the second inlet 16 is removed farther from the first side 13 of the test combustion chamber 12 than the first inlet 14.

As an alternative to the exemplary embodiment, the fuel G and the air L could also be mixed in a mixer outside the test combustion chamber 12.

An ignition means, i.e., an ignition electrode 17 in accordance with the example, is arranged in the region of the second inlet 16 in order to be able to generate a test burner flame 18 in the test combustion chamber 12. In modification of the exemplary embodiment shown here, a catalyst could also be installed in the test combustion chamber 12 in order to trigger an oxidation of the mixture of gaseous fuel G and air L.

At a distance from the first side 13, an exhaust gas duct 22 leads out of the test combustion chamber 12. In the exemplary embodiment, the orifice of the exhaust gas duct 22 is arranged on the second side 23 of the test combustion chamber 12, said second side being opposite the first side 13.

An air ratio sensor 24 is installed in the exhaust gas duct 22. The air ratio sensor 24 generates an air ratio signal SL. The air ratio signal SL is transmitted to a test control unit 25. Depending on the received air ratio signal SL, the test control unit 25 generates at least one setting signal S1, S2 for actuating the test supply unit 15. Referring to the exemplary embodiment described here, the test control unit 25 generates a first setting signal S1 as well as a second setting signal S2 for the test supply unit 15. The proportion and/or amount of the air L and/or of the gaseous fuel G being introduced into the test combustion chamber 12 can be adjusted via the at least one setting signal S1, S2.

Referring to the exemplary embodiment in accordance with FIG. 1, the first setting signal S1 actuates a first setting means 26, for example a governable proportional valve, in order to adjust the supplied amount of air L being supplied to the test combustion chamber 12. In accordance with the example, a second setting means 27, for example a governable proportional valve, is actuated by means of the second setting signal S2 in order to adjust the amount of gaseous fuel G that is supplied to the test combustion chamber 12. In this manner, the proportion of air L or gaseous fuel G, as well as the total amount of the gas mixture supplied to the test combustion chamber 12 can be variably adjusted. As an alternative to the exemplary embodiment, the amount of supplied gaseous fuel G or of supplied air L could also be prespecified so as to be invariably constant so that only the respective other component of the mixture could be adjusted for changing the proportions via an setting means 26 or 27.

As an alternative or an addition to the governable proportional valve, the first setting means 26 could also comprise a governable blower or the like.

A calorific value sensor arrangement 30 is provided in the test combustion chamber 12. The calorific value sensor arrangement 30 comprises at least one ionization sensor 31 that generates an ionization signal and transmits said signal to a determination unit 23. In the exemplary embodiment, the ionization signal is generated by the ionization current I. As is schematically shown in FIG. 1, it is also possible for several ionization sensors 31 to be present, each of said sensors transmitting a separate ionization signal I to the determination unit 32. As a result of this, the ionization can be measured at various locations in the test combustion chamber 12 and, in particular, in the region of the test combustion flame 18. A single ionization sensor 31 is sufficient, said sensor being arranged, in particular, in the region of the test burner flame 18.

Referring to the exemplary embodiment described here, the calorific value sensor arrangement 30 further comprises at least one temperature sensor 33. Preferably, the temperature sensor 33 is also installed in the region of the test burner flame 18 in the test combustion chamber 12. The temperature sensor 33 delivers a temperature signal ST to the determination unit 32.

As is schematically illustrated in FIG. 1, it is possible in a preferred embodiment to additionally make signals or parameters available to the determination unit 32. Preferably, one or more of the following signals are transmitted to the determination unit: the air ratio number signal SL and/or the first setting signal S1 and/or the second setting signal S2.

Based on the at least one setting signal S1, S2, the actually set burner performance P of the test burner 11 can be determined or at least estimated. Inasmuch as the burner performance P affects the ionization current I it is advantageous to supply a signal describing the burner performance P to the determination unit 32. In accordance with the example, this is achieved by the transmission of the at least one setting signal S1, S2. Alternatively, it is also possible to maintain the burner performance P constant at all times, so that the burner performance P can be prespecified to the determination unit 32 and need not be determined by the transmission of a signal.

The calorific value parameter H is determined by the determination unit 32 based on the transmitted signals and, for example, the ionization signal I, the temperature signal ST, as well as the at least one setting parameter S1, S2. To accomplish this in accordance with the example, a characteristic map for determining the calorific value parameter H based on the input signals I, R, S1 and S2 is stored in a memory 34 of the determination unit 32. The characteristic map can be stored in the memory 34 in form of one or several tables or matrices.

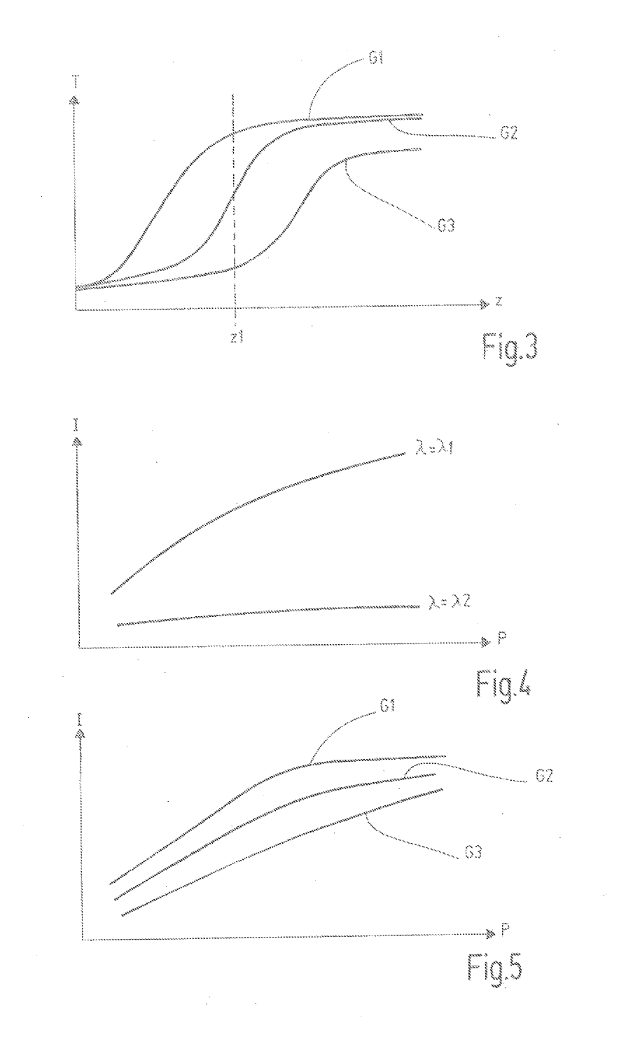

FIGS. 3 through 5, hereinafter, are used to explain the function of the apparatus 10 for the determination of the calorific value parameter H hereinafter.

At the start of operation, the test supply unit 15 is adjusted in such a manner that a prespecified amount of gaseous fuel G and air L is introduced into the test combustion chamber 12. The starting value for adjusting the test supply unit 15 may be provided by the at least one setting parameter S1, S2 that was used last during the previous operation of the apparatus 10. This value for the at least one setting value S1, S2 can be stored in the memory 34 of the determination unit 32 and be made available to the test control unit 25. The starting setting of the test supply unit 15 may also be performed, alternatively or additionally, by means of the last-determined calorific value parameters H. To do so, the calorific value parameter H that has been actually determined can be stored in the memory 24 during operation of the apparatus 10.

Subsequently, the test burner flame 18 is ignited by the ignition electrode 17. The test supply unit 15 is actuated via the test control unit 25 in such a manner that the air ratio λ corresponding to the air ratio signal SL corresponds to a nominal air ratio value. This nominal air ratio value is preferably greater than or equal to one.

As has been schematically shown in FIG. 4, the ionization current I measured by the ionization sensor 31 is a function of the set air ratio λ. The values of the ionization current I are evaluated in the determination unit 32 in particular only when a prespecified air ratio λ has been set via the test control unit 25 and the test supply arrangement 15. Alternatively, it is also possible to allow fluctuations of the air ratio λ and to transmit the air ratio signal SL to the determination unit 32, so that the respectively actual air ratio λ can be taken into account in the determination of the calorific value parameter H. Furthermore, it is possible to adjust several prespecified nominal air ratio values in chronological order and to perform, at each of the air ratio setpoint values, an evaluation of the at least one sensor signal of the calorific value sensor arrangement and, in particular, of the ionization current I and the temperature signal ST, taking into consideration the current test burner performance P, and to determine the calorific value parameter H therefrom.

FIG. 3 shows a graph wherein the temperature T is a function of the distance z from the base or core of the flame and, for example, on the second inlet 16. This graph shows that, at certain distances Z, it is easier to distinguish among different compositions G1, G2, G3 of the gaseous fuel G. As the graph of FIG. 3 schematically shows, the distance z=z1 was selected (see also FIG. 1) because, in this range, the differences of temperature T for the various compositions G1, G2, G3 of the gaseous fuel G can be distinguished clearly.

FIG. 5 is a schematic graph of the ionization current I as a function of the performance P of the test burner 12. FIG. 5 shows that different compositions G1, G2, G3 of the gaseous fuel G result in different ionization currents I with a known or prespecified burner performance P of the test burner 11.

These different compositions G1, G2, G3 of the gaseous fuel G have different calorific values. Therefore, it is possible, in a characteristic map of the determination unit 32, to allocate, respectively, one calorific value parameter H to the measured ionization currents I and the measured temperatures T, optionally taking into account the current air ratio λ, as well as the actual burner performance P. As mentioned, the air ratio λ and the burner performance P can either be set prespecified at a specific value or said value can be variable. In the latter case, respectively one signal defining the air ratio λ or the actual burner performance P can be transmitted to the determination unit 32 for the consideration of the current air ratio λ or the burner performance P.

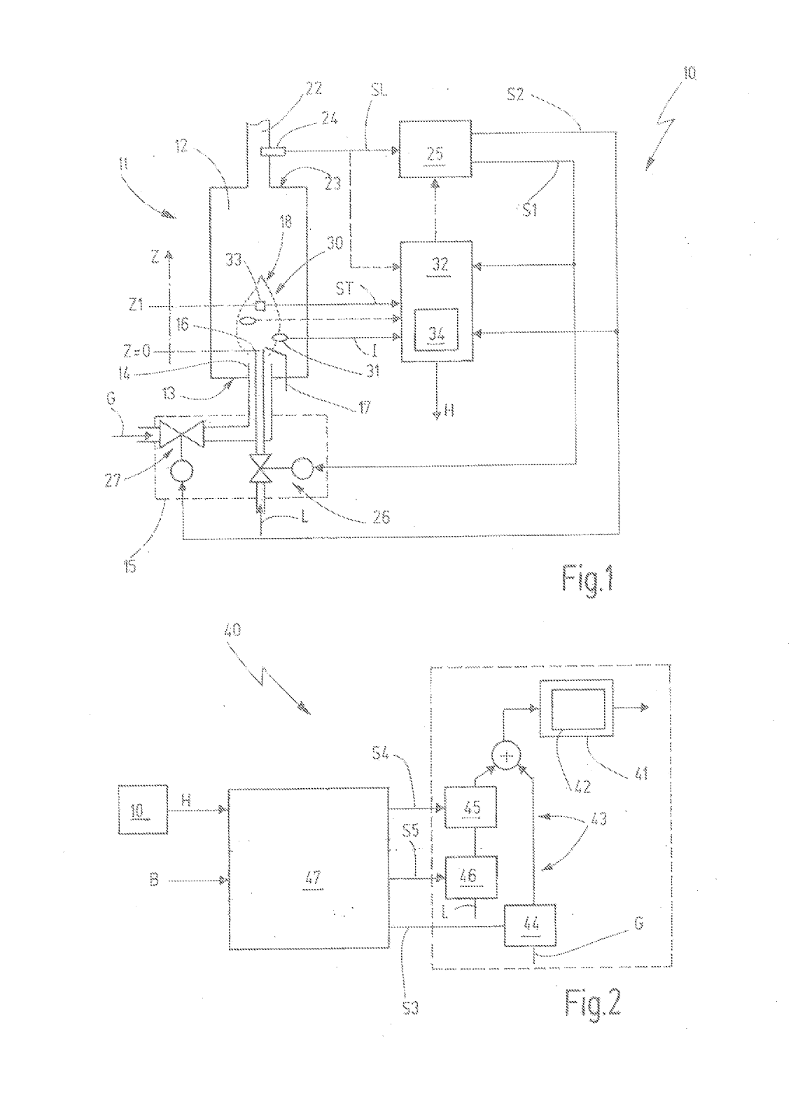

The apparatus 10 in accordance with FIG. 1 can be advantageously installed in a gas-powered arrangement 40 (FIG. 2) comprising a main burner 41, said main burner comprising a main combustion chamber 42. The gas-powered arrangement comprises a controllable main supply arrangement 43. The main supply arrangement 43 comprises a third setting means 44 by means of which the amount of gaseous fuel G supplied to the main combustion chamber 42 can be adjusted. Furthermore, the main supply arrangement 43 possesses a fourth setting means 45 and/or a fifth setting means 46. The amount of an oxygen-containing gas, for example air L, that is supplied to the main combustion chamber 42 is adjusted by means of the fourth setting means 45 and/or the fifth setting means 46. As has been described in conjunction with the apparatus 10 in accordance with FIG. 1, the third setting means 44 and the fourth setting means 45 may be represented by governable proportional valves. Alternatively or additionally, a fifth setting means 46, for example, a governable blower, may be provided for influencing the amount of air. The gaseous fuel G and the air L can be mixed, as schematically illustrated by FIG. 2, outside the main combustion chamber 42 or, alternatively, also inside the main combustion chamber 42.

Each setting means 44, 45, 46 of the main supply arrangement 43 is allocated a corresponding setting signal S3, S4, S5 of a main control unit 47. The at least one setting signal S3, S4, S5 generated by the main control unit 47 in order to actuate the main supply unit 43 is determined as a function of the calorific value parameter H of the apparatus 10. In addition, the determination of the at least one setting signal S3, S4, S5 of the main control unit 47 can be a function of additional signals or parameters, for example, a control signal B, by means of which the operating mode or the burner performance of the main burner 41 is prespecified. The control signal B, for example, may be set by an operator or also automatically by a controller or a control unit. Furthermore, the main burner 41 may also be allocated sensors whose sensor signals are transmitted to the main control unit 47 and can be used for the determination of the at least one setting signal S3, S4, S5. For example, it is also possible for an air ratio sensor to be present in an exhaust duct of the main burner 41, so that the at least one setting signal S3, S4, S5 is adjusted via the main control unit 47 in order to achieve a prespecified air ratio.

For example, a characteristic map may be stored in the main control unit 47, said characteristic map being a function of at least the calorific value parameter H, and with the use of which one corresponding value for the at least one setting signal S3, S4, S5, respectively, generated by the main control unit 47 is obtained. As in the example, the characteristic map also takes into consideration the control signal B as an input parameter and/or as an additional parameter such as, for example, an air ratio signal of an air ratio sensor in the exhaust gas duct of the main burner 41.

Consequently, a control or regulation of the main burner 41 adapted to the quality or the calorific value of the gaseous fuel G can be accomplished.

The determined calorific value parameter H can also be used for other applications. For example, it is also possible to transmit the calorific value parameter H of the gaseous fuel G to an arrangement in order to determine the energy consumption. In that case, the energy consumption is not determined taking into consideration the amount of gas that has been used but is determined also taking into consideration the calorific value associated with a specific amount of gas. This application represents another stand-alone, independent aspect of the invention.

The invention relates to a method and an apparatus for the determination of a calorific value parameter H describing the calorific value of a gaseous fuel G. The invention also relates to a gas-powered arrangement 40 that uses a method according to the invention and an apparatus 10 according to the invention. The apparatus 10 comprises a test burner 11 with a test combustion chamber 12. An air ratio sensor 24 is arranged in an exhaust gas duct 22 of the test burner 11 and measures an air ratio signal SL that corresponds to the air ratio λ of the exhaust gas. As a function of the received air ratio signal SL, at least one setting signal S1, S2 is generated for a test supply unit 15 via a test control unit 25. The at least one setting signal S1, S2 controls the amount and/or the proportion of a gaseous fuel G or an oxygen-containing gas L that is supplied to the test combustion chamber 12. The mixture of fuel G and the oxygen-containing gas L is combusted or oxidized in the test combustion chamber 12. A calorific value sensor arrangement 30 is provided in the combustion chamber 12. This sensor arrangement comprises at least one ionization sensor 31 and, preferably, also a temperature sensor 33. The at least one sensor signal I or T of the calorific value sensor arrangement is transmitted to a determination unit 32. There, a characteristic map of these parameters is preferably used for determining a calorific value parameter H. For the determination of the calorific value parameter H, it is also optionally possible for a signal defining the air ratio λ and/or a signal defining the burner performance P of the test burner 11 to be transmitted to the determination unit 32 and to be taken into consideration.

### LIST OF REFERENCE SIGNS

10 Apparatus
11 Test burner
12 Test combustion chamber
13 First side of the test combustion chamber
14 First Inlet
15 Test supply unit
16 Second inlet
17 Ignition electrode
18 Test burner flame
22 Exhaust gas duct
23 Second side of the test combustion chamber
24 Air ratio sensor
25 Test control unit
26 First setting means
27 Second setting means
30 Calorific value sensor arrangement
31 Ionization sensor
32 Determination unit
33 Temperature sensor
34 Memory
40 Gas-powered arrangement
41 Main burner
42 Main combustion chamber
43 Main supply arrangement
44 Third setting means
45 Forth setting means
46 Fifth setting means
47 Main control unit
λ Air ratio
B Control signal
G Gaseous fuel
G1 First composition of the gaseous fuel
G2 Second composition of the gaseous fuel
G3 Third composition of the gaseous fuel
H Calorific value parameter
I Ionization signal
S1 First setting signal
S2 Second setting signal
S3 Third setting signal
S4 Fourth setting signal
S5 Fifth setting signal
SL Air ratio signal
ST Temperature signal

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. Apparatus for the determination of a calorific value parameter (H) for the calorific value of a gaseous fuel (G), comprising
a test combustion chamber (12) and an exhaust gas duct (22) leading out of the test combustion chamber (12), in which case an air ratio sensor (24) is arranged in said combustion chamber (12),
a controllable test supply unit (15), by means of which the amount of the gaseous fuel (G) and/or the amount of the oxygen-containing gas (L) is adjusted, in which case said amounts are introduced, respectively, in the test combustion chamber (12),
a test control unit (25) to which the air ratio signal (SL) is transmitted and which, as a function of the air ratio signal (SL), generates at least one setting signal (S1, S2), said signal being used to actuate the test supply unit (15), so that the air ratio (λ) corresponds to a prespecified nominal air ratio value,
a calorific value sensor arrangement (30) located in the test combustion chamber (12), said arrangement comprising at least one ionization sensor (31),
a determination unit (32), to which the ionization signal (I) of the ionization sensor (31) is transmitted, wherein the determination unit (32) determines the calorific value parameter (32) as a function of the ionization signal (I), said parameter indicating the calorific value of the gaseous fluid (G).

2. Apparatus as in claim 1, characterized in that the air ratio signal (SL) is also transmitted to the determination unit (32) and that the determination unit (32) additionally determines the calorific value parameter (H) as a function of the air ratio signal (SL).

3. Apparatus as in claim 1, characterized in that the at least one setting signal (S1, S2) is also transmitted to the determination unit (32) determination unit (32), and that the determination unit (32) additionally determines the calorific value parameter (H) as a function of the setting signal (S1, S2).

4. Apparatus as in claim 1, characterized in that the calorific value sensor arrangement (30) comprises at least one temperature sensor, and that the temperature signal (ST) of the temperature sensor (33) is transmitted to the determination unit (32), said unit additionally determining the calorific value parameter (H) as a function of the temperature signal (ST).

5. Apparatus as in claim 1, characterized in that the determination unit (32) comprises a memory (34) in which a determination specification for the determination of the calorific value parameter (H) is prespecified as a function of the ionization signal (I) and of at least one additional parameter (T, SL, S1, S2).

6. Apparatus as in claim 1, characterized in that a test burner flame (18) is generated in the test combustion chamber (12).

7. Apparatus as in claim 1, characterized in that, for the determination of the calorific value parameter (H) in the determination arrangement (32), the temperature signal (ST) and/or the ionization signal (I) is detected at a prespecified value or at several prespecified values for the air ratio (λ).

8. Apparatus as in claim 1, characterized in that several ionization sensors (31) are arranged at various locations inside the test combustion chamber (12).

9. Gas-powered arrangement (40) comprising a controllable main supply arrangement (43) by means of which the amount of a gaseous fuel (G) and the amount of an oxygen-containing gas (L) conducted into a main combustion chamber (42) are adjusted, said arrangement comprising
an apparatus (10) for the determination of a calorific value parameter (H) in accordance with one of the previous claims, and
a main control unit (47) that generates at least one setting signal (S3, S4, S5) as a function of the calorific value parameter (H) in order to actuate the main supply arrangement (43).

10. Gas-powered arrangement as in claim 9, characterized in that the main control unit (47) is disposed to enable the supply of gaseous fuel (G) via the main supply unit (43) into the main combustion chamber (42) only when a calorific value parameter (H) has been transmitted by the apparatus (10) for the determination of a calorific value parameter (H).

11. Method for the determination of a calorific value parameter (H) for the calorific value of a gaseous fuel (G) with the use of an apparatus (10) comprising
a test combustion chamber (12) and an exhaust gas duct (22) leading out of the test combustion chamber (12), whereby an air ratio sensor (24) is arranged in said exhaust gas duct,
a controllable test supply unit (15), by means of which the amount of the gaseous fuel (G) and/or the amount of an oxygen-containing gas (L) is adjusted, said amounts being respectively conducted into the test combustion chamber (12),
a test control unit (25) to which the air ratio signal (SL) is transmitted and which generates at least one setting signal (S1, S2) for the test supply unit (15) as a function of the air ratio signal (SL),
a calorific value sensor arrangement (30) located in the test combustion chamber (12), said arrangement comprising at least one ionization sensor (31), and
a determination unit (32) to which the ionization signal (I) of the ionization sensor (31) is transmitted, wherein
the test supply unit (15) is actuated in such a manner that the air ratio (λ) corresponds to a prespecified nominal air ratio (λ),
the determination unit (32) determines the calorific value parameter (H) as a function of the ionization signal (I), said calorific value parameter specifying the calorific value of the gaseous fuel.

12. Method as in claim 11, characterized in that at least the actually determined value of the fuel parameter (H) is stored in a memory.

13. Method as in claim 12, characterized in that the stored value of the fuel parameter (H) is used during the subsequent start of the combustion or oxidation in the test combustion chamber (12) for adjusting the amount of gaseous fuel (G) and/or the amount of an oxygen-containing gas (L), said amounts being respectively introduced into the test combustion chamber (12).
